(19) United States
(12) Patent Application Publication
(10) Pub. No.: US 2021/0137665 A1
(43) Pub. Date: May 13, 2021

(54) LIGHT-EMITTING INTRAORAL DEVICE

(71) Applicant: SUNSTAR SUISSE S.A., Etoy (CH)

(72) Inventors: Ray BURKE, Whitechurch, Co. Cork (IE); Haiyang LI, Cork (IE); Toru SAITO, Buffalo Grove, IL (US); Leoncio Angel GONZALEZ, Winfield, IL (US); Josh VICARI, Schaumburg, IL (US)

(21) Appl. No.: 16/959,656

(22) PCT Filed: Jan. 11, 2019
(86) PCT No.: PCT/US2019/013331

§ 371 Date: Jul. 1, 2020

Related U.S. Application Data
(60) Provisional application No. 62/616,706, filed on Jan. 12, 2018.

(51) Int. Cl. A61C 19/06; A61N 5/06

(52) U.S. Cl. CPC A61C 19/06; A61N 2005/0606; A61N 2005/0663; A61N 5/0603

(57) ABSTRACT

An intraoral device comprises a body including a lingual wall, a labial wall, and a bite surface extending between the lingual wall and the labial wall, wherein the lingual wall, the labial wall, and the bite surface define at least one channel; and a light source coupled to the body and configured to illuminate at least a portion of the at least one channel.

### BACKGROUND

The present disclosure relates generally to an intraoral dental care device. More specifically, the present disclosure relates to an intraoral device that applies light to teeth and gums.

The human oral cavity includes several environmental conditions that may encourage the growth of harmful bacteria. For example, the human oral cavity provides a warm, humid environment with complex surfaces ready for colonization, and a regular supply of organic material on which bacteria or other microorganisms may feed. Such bacteria or other microorganism may cause undesirable conditions such as gingivitis, periodontitis, halitosis, and the like.

Accordingly, there exists a need for a device and method for killing harmful bacteria and other microorganisms in the oral cavity.

### SUMMARY OF THE DISCLOSURE

Various aspects of the present disclosure relate to light-emitting intraoral devices and methods of making and/or using the same.

In one exemplary aspect of the present disclosure, there is provided an intraoral device, comprising: a body including a lingual wall, a labial wall, and a bite surface extending between the lingual wall and the labial wall, wherein the lingual wall, the labial wall, and the bite surface define at least one channel; and a light source coupled to the body and configured to illuminate at least a portion of the at least one channel.

In another exemplary aspect of the present disclosure, there is provided an intraoral therapy method, comprising: providing an intraoral device, the intraoral device including: a body including a lingual wall, a labial wall, and a bite surface extending between the lingual wall and the labial wall, wherein the lingual wall, the labial wall, and the bite surface define at least one channel, and a light source coupled to the body; and illuminating at least a portion of the at least one channel via the light source.

In another exemplary aspect of the present disclosure, there is provided a method of forming an intraoral device, comprising: forming a body including a lingual wall, a labial wall, and a bite surface extending between the lingual wall and the labial wall, wherein the lingual wall, the labial wall, and the bite surface define at least one channel; and coupling a light source to the body such that the light source may illuminate at least a portion of the at least one channel.

This disclosure can be embodied in various forms. The foregoing summary is intended solely to provide a general description of various aspects of the present disclosure, and does not limit the scope of the disclosure in any way.

### BRIEF DESCRIPTION OF THE DRAWINGS

These and other more detailed and specific features of various aspects of the present disclosure are more fully disclosed in the following description, reference being had to the accompanying drawings, in which:

FIG. 1 illustrates a perspective view of an exemplary intraoral device according to various aspects of the present disclosure;

FIG. 2 illustrates an exemplary light source for use with the intraoral device of FIG. 1;

FIG. 3 illustrates another perspective view of the intraoral device of FIG. 1;

FIG. 4 illustrates a top plan view of the intraoral device of FIG. 1;

FIG. 5 illustrates an exemplary exposure area according to various aspects of the present disclosure;

FIG. 6 illustrates a perspective view of another exemplary intraoral device according to various aspects of the present disclosure;

FIG. 7 illustrates an exemplary light emitting portion for use with the intraoral device of FIG. 6;

FIG. 8 illustrates another perspective view of the intraoral device of FIG. 6;

FIG. 9 illustrates a top plan view of the intraoral device of FIG. 6;

FIG. 10 illustrates a perspective view of a substructure for another exemplary intraoral device according to various aspects of the present disclosure; and

FIG. 11 illustrates a perspective view of an overmold for use with the substructure of FIG. 10.

### DETAILED DESCRIPTION

In the following description, numerous details are set forth. It will be readily apparent to one skilled in the art that these specific details are merely exemplary and not intended to limit the scope of this disclosure.

Intraoral devices according to the present disclosure utilize light (such as blue light) to kill harmful bacteria in the oral cavity, thus noninvasively improving the prevention and treatment of gum disease such as gingivitis and periodontitis, and bad breath.

Intraoral devices according to the present disclosure are designed as a supplement to daily oral hygiene habits, including invasive techniques such as brushing and flossing. The intraoral devices are to be used at any point during the day, and preferably used once or twice daily (for example, at a user's home) for a period of time (for example, up to three minutes).

FIGS. 1 and 3-4 illustrate an exemplary intraoral device 100 in several views. The intraoral device 100 includes a body including a handle 110, a bite surface 120, an inner wall 130 (an example of a “lingual wall”), and an outer wall 140 (an example of a “labial wall”). The bite surface 120, the inner wall 130, and the outer wall 140 define at least one channel. The intraoral device 100 is generally sized for a human oral cavity such that, in use, the user grips the bite surface 120 with the user's teeth disposed in the channel between the inner wall 130 and the outer wall 140. Thus, the intraoral device 100 remains in place during use.

As illustrated in FIG. 1, the inner wall 130 includes a first groove 131a located in a portion which extends above the bite surface 120 and a second groove 131b located in a portion which extends below the bite surface 120. The outer wall 140 includes a third groove 141a located in a portion which extends above the bite surface 120 and a fourth groove 141b located in a portion which extends below the bite surface 120. In use, the first groove 131a faces the lingual surface of the upper teeth and gums, the second groove 131b faces the lingual surface of the lower teeth and gums, the third groove 141a faces the buccal and labial surfaces of the upper teeth and gums, and the fourth groove 141b faces the buccal and labial surfaces of the lower teeth and gums. Each groove 131a-b and 141a-b is adapted to receive a corresponding light source 132 therein, one of which is illustrated in FIG. 2. The light sources 132 respectively include a flexible circuit board 133 and at least one light emitting device 134, such as a light-emitting diode (LED). In one example, the bite surface 120 includes an embedded sensor, such as a pressure sensor, a temperature sensor, a capacitive sensor, or an optical sensor, which determines that a user has placed the intraoral device 100 in his or her mouth and causes the light sources 134 to emit light. For example, the temperature sensor may detect that the temperature is above a predetermined threshold corresponding to a temperature of the oral cavity; the capacitive sensor may detect that the humidity corresponds to an oral environment, and the like. In another example, the handle 110 includes a power switch that allows the user to determine when the light sources 134 are to begin emitting.

The handle 110 may include a port 111 to receive a power cable therein. The port 111 may comply with a communication standard; for example, Universal Serial Bus (USB). In one example, the handle 110 further includes a rechargeable battery 112 electrically connected to the port 111 so as to allow the intraoral device 100 to be charged when not in use. The rechargeable battery 112 may be one or more supercapacitors; for example, two supercapacitors. In addition or as an alternative to the port 111, the handle may include circuitry capable of wireless charging, such as an inductive coil or loop. The circuitry may be in compliance with one or more wireless charging standards; for example, Near Field Communication (NFC), Qi, and the like. In another example, the handle 110 does not include a battery and instead includes electrical connections between the light sources 132 and the port 111 so as to allow the intraoral device 100 to be connected to an external power source during use. The handle 110 may further include a controller 113 to control the timing and operation of the intraoral device 100. The controller 113 may be configured to control the light sources 132 according to an illumination pattern data. For example, the controller 113 may be configured to automatically turn the light sources 132 off after a predetermined exposure time has elapsed.

Moreover, the controller 113 may be configured to detect the presence of a particular biomarker or other bacterial indicator (such as via a sensor included in the bite surface 120) and modify the treatment parameters (such as wavelength, intensity, and/or time) accordingly so as to treat a corresponding condition indicated by the biomarker. The wavelength, intensity, exposure time, and the like are selected such that, in use, only periodontal pathogens are killed while non-pathogenic bacteria remain substantially unharmed. Examples of periodontal pathogens include P. gingivalis, P. intermedia, F. nucleatum, and the like. Examples of non-pathogenic bacteria include S. oralis and the like.

The predetermined exposure time may be from about 60 to 600 s; preferably, from about 60 to 180 s. Moreover, in examples where the handle 110 includes the rechargeable battery 112, the controller 113 may be configured to determine whether the power cable is connected to the port 111 and, if the power cable is connected to the port 111, prevent operation of the intraoral device 100 to reduce the risk of electrical shock. The handle 110 may also include communication circuitry, such as a Wi-Fi or Bluetooth antenna, a low-power radio (LPR), and the like, so that the intraoral device 100 is capable of communication with a base station, a user's smartphone or tablet, or the like. The communication circuitry may be connected to the port 111 to allow a wired communication, for example via USB.

The handle 110, the bite surface 120, the inner wall 130, and the outer wall 140 may be formed with a unitary construction of a flexible or rigid biocompatible material. The biocompatible material may be an elastomeric or hard plastic, such as a polymer or any other material that exhibits low or no odor, taste, or toxicity. Additionally, the handle 110, the bite surface 120, the inner wall 130, and the outer wall 140 may be formed using a two-shot molding process, a three-shot molding process, a 3D-printing process, and the like. Where the handle 110, the bite surface 120, the inner wall 130, and the outer wall 140 are formed using multiple materials, the materials may include a biocompatible material molded over a frame formed of a material having a higher rigidity. The frame may be a wire or plastic substructure. In some examples, the outermost material may be formed of a soft elastomer to provide increased user comfort. In some examples, the outermost material may be a disposable shell.

The light sources 132 are configured to emit light with a wavelength and intensity that kills harmful oral bacteria. For example, the light sources 132 are configured to emit light in a wavelength range from about 400 nm to 700 nm. Preferably, the light sources 132 are configured to emit light with a wavelength, corresponding to blue light, of about 450 nm to 470 nm. Additionally, the light sources 132 are configured to emit light with an intensity such that the user's teeth and gums receive an irradiance from, for example, about 10 mW/cm² to 1000 mW/cm². Preferably, the irradiance is about 25 mW/cm² to 100 mW/cm². Moreover, the light sources 132 may each be configured to emit light with substantially the same wavelength, or alternatively may be configured to emit light with wavelengths different from one another. That is, in one example, each of the light sources 132 may be configured to emit light in a wavelength range from about 450 nm to 470 nm. In another example, a first (blue) subset of the light sources 132 may be configured to emit light in a wavelength range about 450 nm to 470 nm, whereas a second (red) subset of the light sources 132 may be configured to emit light in a wavelength range from about 650 nm to 670 nm. Where the light sources 132 are configured to emit different wavelengths, the individual light sources 132 may be arranged in an alternating manner such that each light source 132 in the first subset is disposed between adjacent light sources 132 in the second subset, and vice versa.

Additionally, the light sources 132 are configured to irradiate a predetermined exposure area 500 including a user's teeth surfaces above the gum line (i.e., tooth enamel), the area between gums and teeth (i.e., the sulcular epithelium), and the margins of gums (i.e., the gingival margin and the oral epithelium). FIG. 5 illustrates a partial view of the exposure area, including an upper exposure area 501 and a lower exposure area 502. FIG. 5 shows only an area including a user's incisor teeth 511 and gums 512; however, in practice the upper and lower exposure areas 501-502 extend from the incisors to the third molars. Where the intraoral device 100 is sized for a child, the upper and lower exposure areas 501-502 may extend from the incisors to the second molars. Preferably, the predetermined exposure area 500 is about 25 cm², such that the upper exposure area 501 and the lower exposure area each have a height of about 5 mm.

FIGS. 6 and 8-9 illustrate another exemplary intraoral device 600. The intraoral device 600 includes a body including a handle 610, a bite surface 620, an inner wall 630 (an example of a “labial wall”), and an outer wall 640 (an example of a “lingual wall”). The bit surface 620, the inner wall 630, and the outer wall 640 define at least one channel. Similar to the intraoral device 100 described above, the intraoral device 600 is generally sized for a human oral cavity such that, in use, the user grips the bite surface 620 with the user's teeth disposed in the channel between the inner wall 630 and the outer wall 640. Thus, the intraoral device 600 remains in place during use.

As illustrated in FIG. 6, the inner wall 630 includes a first groove 631a located in a portion which extends above the bite surface 620 and a second groove 631b located in a portion which extends below the bite surface 620. The outer wall 640 includes a third groove 641a located in a portion which extends above the bite surface 620 and a fourth groove 641b located in a portion which extends below the bite surface 620. In use, the first groove 631a faces the lingual surface of the upper teeth and gums, the second groove 631b faces the lingual surface of the lower teeth and gums, the third groove 641a faces the buccal and labial surfaces of the upper teeth and gums, and the fourth groove 641b faces the buccal and labial surfaces of the lower teeth and gums. Each groove 631a-b and 641a-b is adapted to receive a corresponding light emitting portion therein. An example of the light emitting portion configuration is illustrated in FIG. 7.

In contrast with the intraoral device 100, which as illustrated directly utilized LED light sources, the intraoral device 600 as illustrated utilizes a light pipe configuration. That is, as shown in FIG. 7, the light emitting portions are constituted by a light fiber 632 that receives an input light beam at one end or both ends thereof from a light source 614, described in more detail below, and diffuses the light throughout its length. In order to appropriately diffuse the light input at one or both ends along the length of the light fiber 632, the light fiber 632 may include surface features. For example, the light fiber 632 may include asymmetrical or symmetrical cuts or notches, an appropriately-shaped transparent window in a non-transparent coating,

The handle 610 includes the at least one light source 614. As particularly illustrated in FIGS. 6 and 8-9, the handle 610 includes four light sources 614. Preferably, the light sources 614 are each constituted by an LED, a laser diode, a superluminescent diode (SLED/SLD), or the like. An end of the light fiber 632 is in optical communication with one light source 614, such that light from the light source 614 enters the light fiber 632 so as to be incident on the user's teeth and gums across the length of the light fiber 632. The light fibers 632 and the light sources 614 may be provided in a one-to-one correspondence, such that each light fiber 632 receives light from a light source 614 at one end thereof, or in a one-to-two correspondence, such that each light fiber 632 receives light from one light source 614 at one end thereof and from another light source 614 at the other end thereof. A total of one, two, or four light fibers 632 may be used. For example, a single light fiber 632 may extend through all four grooves 631a-b and 641a-b; a first light fiber 632 may extend through two grooves 631a-b and a second light fiber 632 may extend through two grooves 641a-b; a first light fiber 632 may extend through grooves 631a and 641a and a second light fiber 632 may extend through grooves 631b and 641b; or a first light fiber 632 may extend through groove 631a, a second light fiber 632 may extend through groove 641a, a third light fiber 632 may extend through groove 631b, and a fourth light fiber 632 may extend through groove 641b. Thus, because either one or two light sources 614 may be provided for each light fiber 632, a total of one, two, four, or eight light sources 614 may be provided.

In one example, the bite surface 620 includes an embedded sensor, such as a pressure sensor, a temperature sensor, a capacitive sensor, or an optical sensor, which determines that a user has placed the intraoral device 600 in his or her mouth and causes the light sources 614 to emit light. For example, the temperature sensor may detect that the temperature is above a predetermined threshold corresponding to a temperature of the oral cavity; the capacitive sensor may detect that the humidity corresponds to an oral environment, and the like. In another example, the handle 610 includes a power switch that allows the user to determine when the light sources 614 are to begin emitting. In one example, the handle 610 includes an additional heat dissipating portion (not shown), such as a heat sink, to direct heat generated by the light sources 614 away from the user. In another example, the handle 610 may be formed of a material that itself dissipates heat, such that the handle 610 itself acts as a heat sink.

The handle 610 may include a port 611 to receive a power cable therein. The port 611 may comply with a communication standard; for example, USB. In one example, the handle 610 further includes a rechargeable battery 612 electrically connected to the port 611 so as to allow the intraoral device 600 to be charged when not in use. The rechargeable battery 612 may be one or more supercapacitors; for example, two supercapacitors. In addition or as an alternative to the port 111, the handle may include circuitry capable of wireless charging, such as an inductive coil or loop. The circuitry may be in compliance with one or more wireless charging standards; for example, NFC, Qi, and the like. In another example, the handle 610 does not include a battery and instead includes electrical connections between the light sources 614 and the port 611 so as to allow the intraoral device 600 to be connected to an external power source during use. The handle 610 may further include a controller 613 to control the timing and operation of the intraoral device 600. The controller 613 may be configured to control the light sources 614 according to an illumination pattern data. For example, the controller 613 may be configured to automatically turn the light sources 614 off after a predetermined exposure time has elapsed.

Moreover, the controller 613 may be configured to detect the presence of a particular biomarker or other bacterial indicator (such as via a sensor included in the bite surface 620) and modify the treatment parameters (such as wavelength, intensity, and/or time) accordingly so as to treat a corresponding condition indicated by the biomarker. The wavelength, intensity, exposure time, and the like are selected such that, in use, only periodontal pathogens are killed while non-pathogenic bacteria remain substantially unharmed. Examples of periodontal pathogens include P. gingivalis, P. intermedia, F. nucleatum, and the like. Examples of non-pathogenic bacteria include S. oralis and the like.

The predetermined exposure time may be from about 60 to 600 s; preferably, from about 60 to 180 s. Moreover, in examples where the handle 610 includes the rechargeable battery 612, the controller 613 may be configured to determine whether the power cable is connected to the port 611 and, if the power cable is connected to the port 611, prevent operation of the intraoral device 600 to reduce the risk of electrical shock. The handle 610 may also include communication circuitry, such as a Wi-Fi or Bluetooth antenna, LPR, and the like, so that the intraoral device 600 is capable of communication with a base station, a user's smartphone or tablet, or the like. The communication circuitry may be connected to the port 111 to allow a wired communication, for example via USB.

The handle 610, the bite surface 620, the inner wall 630, and the outer wall 640 may be formed with a unitary construction of a flexible or rigid biocompatible material. The biocompatible material may be an elastomeric or hard plastic, such as a polymer or any other material that exhibits low or no odor, taste, or toxicity. Additionally, the handle 610, the bite surface 620, the inner wall 630, and the outer wall 640 may be formed using a two-shot molding process, a three-shot molding process, a 3D-printing process, and the like. Where the handle 610, the bite surface 620, the inner wall 630, and the outer wall 640 are formed using multiple materials, the materials may include a biocompatible material molded over a frame formed of a material having a higher rigidity. The frame may be a wire or plastic substructure. In some examples, the outermost material may be formed of a soft elastomer to provide increased user comfort. In some examples, the outermost material may be a disposable shell.

Similar to the intraoral device 100, the light fibers 632 are configured to emit light (that is, the light received from the light sources 614) with a wavelength and intensity that kills harmful oral bacteria. For example, the light fibers 632 are configured to emit light in a wavelength range from about 400 nm to 600 nm. Preferably, the light fibers 632 are configured to emit light with a wavelength of about 450 nm to 470 nm. Additionally, the light fibers 632 are configured to emit light with an intensity such that the user's teeth and gums receive an irradiance from, for example, about 10 mW/cm² to 1000 mW/cm². Preferably, the irradiance is about 25 mW/cm² to 100 mW/cm². In order to ensure that the light incident on the user's teeth and gums has the above properties, the emission characteristics of the light sources 614 are set to the appropriate values. In other words, the light sources 614 are selected such that the light emitted therefrom, after entering, diffusing through, and exiting the light fibers 632, has the above-described wavelength and irradiance characteristics.

Additionally, the light fibers 632 are configured to irradiate a predetermined exposure area, such as the exposure area 500 described above and including a user's teeth surfaces above the gum line (i.e., tooth enamel), the area between gums and teeth (i.e., the sulcular epithelium), and the margins of gums (i.e., the gingival margin and the oral epithelium).

In accordance with the present disclosure, various elements such as the controller 113 and/or the controller 613 may be implemented using hardware (such as dedicated and/or application-specific circuitry such as a Field-Programmable Gate Array or FPGA), using software, or using a combination of hardware and software. As an example of implementing the present disclosure using software, the requisite processing can be executed by installing a program in which the processing sequence is recorded in the memory of a specialized computer embedded in dedicated hardware, or can be executed by installing the program in a computer that can execute various processing.

For example, the program can be recorded on a hard disk, a solid-state drive (SSD), or a read only memory (ROM) in advance. Alternatively, the program can be temporarily or permanently stored on a removable recording medium such as a flash drive, a semiconductor memory card, and the like. The program may be transferred wirelessly or by wire to the controller 113 and/or the controller 613 from a remote site or server via a network such as a Local Area Network (LAN) or the Internet.

An intraoral device, intraoral therapy method, and method of forming in accordance with the present disclosure may be embodied in any one or more of the following configurations:

(1) An intraoral device, comprising: a body including a lingual wall, a labial wall, and a bite surface extending between the lingual wall and the labial wall, wherein the lingual wall, the labial wall, and the bite surface define at least one channel; and a light source coupled to the body and configured to illuminate at least a portion of the at least one channel.

(2) The intraoral device according to (1), wherein the light source is configured to emit a blue light.

(3) The intraoral device according to (1) or (2), wherein the lingual wall includes a first upper portion which extends above the bite surface and a first lower portion which extends below the bite surface, the labial wall includes a second upper portion which extends above the bite surface and a second lower portion which extends below the bite surface, and the light source includes a first light emitting element coupled to the first upper portion, a second light emitting element coupled to the first lower portion, a third light emitting element coupled to the second upper portion, and a fourth light emitting element coupled to the second lower portion.

(4) The intraoral device according to any one of (1) to (3), further comprising: a controller; and a power source configured to provide a power to the controller and to the light source.

(5) The intraoral device according to (4), wherein the controller is configured to control the light source according to an illumination pattern data.

(6) The intraoral device according to (4) or (5), further comprising a communication circuitry configured to connect to an external device.

(7) The intraoral device according to (6), wherein the communication circuitry is configured to communicate using at least one of a wireless communication or a wired communication.

(8) The intraoral device according to any one of (4) to (7), wherein the power source includes at least one supercapacitor.

(9) The intraoral device according to any one of (4) to (8), wherein the body comprises a handle, and the controller and the power source are housed within the handle.

(10) The intraoral device according to any one of (1) to (9), wherein the body comprises a frame formed of a first material, the lingual wall, the labial wall, and the bite surface are formed of a second material covering the frame, and the first material is more rigid than the second material.

(11) An intraoral therapy method, comprising: providing an intraoral device, the intraoral device including: a body including a lingual wall, a labial wall, and a bite surface extending between the lingual wall and the labial wall, wherein the lingual wall, the labial wall, and the bite surface define at least one channel, and a light source coupled to the body; and illuminating at least a portion of the at least one channel via the light source.

(12) The method according to (11), wherein the illuminating comprises emitting a blue light.

(13) The method according to (11) or (12), wherein the lingual wall includes a first upper portion which extends above the bite surface and a first lower portion which extends below the bite surface, the labial wall includes a second upper portion which extends above the bite surface and a second lower portion which extends below the bite surface, and the light source includes a first light emitting element coupled to the first upper portion, a second light emitting element coupled to the first lower portion, a third light emitting element coupled to the second upper portion, and a fourth light emitting element coupled to the second lower portion.

(14) The method according to any one of (11) to (13), wherein the intraoral device further comprises a controller and a power source, and the method further comprises providing a power to the controller and to the light source from a power source.

(15) The method according to (14), further comprising controlling the light source by the controller according to an illumination pattern data.

(16) The method according to (14) or (15), wherein the intraoral device further comprises a communication circuitry, and the method further comprises connecting to an external device by the communication circuitry.

(17) The method according to any one of (14) to (16), wherein the power source includes at least one supercapacitor.

(18) The method according to any one of (14) to (17), wherein the body comprises a handle, and the controller and the power source are housed within the handle.

(19) The method according to any one of (11) to (18), wherein the body comprises a frame formed of a first material, the lingual wall, the labial wall, and the bite surface are formed of a second material covering the frame, and the first material is more rigid than the second material.

(20) A method of forming an intraoral device, comprising: forming a body including a lingual wall, a labial wall, and a bite surface extending between the lingual wall and the labial wall, wherein the lingual wall, the labial wall, and the bite surface define at least one channel; and coupling a light source to the body such that the light source may illuminate at least a portion of the at least one channel.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain aspects of the present disclosure, and should in no way be construed so as to limit the disclosure.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

What is claimed is:

1. An intraoral device, comprising:
a body including a lingual wall, a labial wall, and a bite surface extending between the lingual wall and the labial wall, wherein the lingual wall, the labial wall, and the bite surface define at least one channel; and
a light source coupled to the body and configured to illuminate at least a portion of the at least one channel.

2. The intraoral device according to claim 1, wherein the light source is configured to emit a blue light.

3. The intraoral device according to claim 1, wherein
the lingual wall includes a first upper portion which extends above the bite surface and a first lower portion which extends below the bite surface,
the labial wall includes a second upper portion which extends above the bite surface and a second lower portion which extends below the bite surface, and
the light source includes a first light emitting element coupled to the first upper portion, a second light emitting element coupled to the first lower portion, a third light emitting element coupled to the second upper portion, and a fourth light emitting element coupled to the second lower portion.

4. The intraoral device according to claim 1, further comprising:
a controller; and
a power source configured to provide a power to the controller and to the light source.

5. The intraoral device according to claim 4, wherein the controller is configured to control the light source according to an illumination pattern data.

6. The intraoral device according to claim 4, further comprising a communication circuitry configured to connect to an external device.

7. The intraoral device according to claim 6, wherein the communication circuitry is configured to communicate using at least one of a wireless communication or a wired communication.

8. The intraoral device according to claim 4, wherein the power source includes at least one supercapacitor.

9. The intraoral device according to claim 4, wherein the body comprises a handle, and the controller and the power source are housed within the handle.

10. The intraoral device according to claim 1, wherein
the body comprises a frame formed of a first material,
the lingual wall, the labial wall, and the bite surface are formed of a second material covering the frame, and
the first material is more rigid than the second material.

11. An intraoral therapy method, comprising:
providing an intraoral device, the intraoral device including:
a body including a lingual wall, a labial wall, and a bite surface extending between the lingual wall and the labial wall, wherein the lingual wall, the labial wall, and the bite surface define at least one channel, and
a light source coupled to the body; and
illuminating at least a portion of the at least one channel via the light source.

12. The method according to claim 11, wherein the illuminating comprises emitting a blue light.

13. The method according to claim 11, wherein
the lingual wall includes a first upper portion which extends above the bite surface and a first lower portion which extends below the bite surface,
the labial wall includes a second upper portion which extends above the bite surface and a second lower portion which extends below the bite surface, and
the light source includes a first light emitting element coupled to the first upper portion, a second light emitting element coupled to the first lower portion, a third light emitting element coupled to the second upper portion, and a fourth light emitting element coupled to the second lower portion.

14. The method according to claim 11, wherein
the intraoral device further comprises a controller and a power source, and
the method further comprises providing a power to the controller and to the light source from a power source.

15. The method according to claim 14, further comprising controlling the light source by the controller according to an illumination pattern data.

16. The method according to claim 14, wherein
the intraoral device further comprises a communication circuitry, and
the method further comprises connecting to an external device by the communication circuitry.

17. The method according to claim 14, wherein the power source includes at least one supercapacitor.

18. The method according to claim 14, wherein the body comprises a handle, and the controller and the power source are housed within the handle.

19. The method according to claim 11, wherein
the body comprises a frame formed of a first material,
the lingual wall, the labial wall, and the bite surface are formed of a second material covering the frame, and
the first material is more rigid than the second material.

20. A method of forming an intraoral device, comprising:
forming a body including a lingual wall, a labial wall, and a bite surface extending between the lingual wall and the labial wall, wherein the lingual wall, the labial wall, and the bite surface define at least one channel; and
coupling a light source to the body such that the light source may illuminate at least a portion of the at least one channel.
